(19) United States
(12) Patent Application Publication
(10) Pub. No.: US 2005/0213035 A1
(43) Pub. Date: Sep. 29, 2005

(54) Virtual image display apparatus for training for correction of strabismus

(75) Inventors: Haruhiko Yoshimeki, Tokyo (JP); Tomoaki Tamura, Tokyo (JP); Hideaki Haraga, Tokyo (JP); Shinri Tanaka, Saitama (JP); Takashi Igarashi, Tokyo (JP); Hiroshi Yamaguchi, Tokyo (JP)

Correspondence Address: MUSERLIAN, LUCAS AND MERCANTI, LLP, 475 PARK AVENUE SOUTH, 15TH FLOOR, NEW YORK, NY, 10016, US

(73) Assignee: Konica Minolta Photo Imaging, Inc.

(21) Appl. No.: 11/077,847

(22) Filed: Mar. 11, 2005

(30) Foreign Application Priority Data
Mar. 25, 2004 (JP) JP2004-090475

(51) Int. Cl. A61B 3/00

(52) U.S. Cl. 351/203

(57) ABSTRACT

An apparatus for displaying a virtual image to perform training for correction of strabismus includes an image capturing section for capturing an outside image such that a normal line-of-sight direction is always positioned at a center of the captured image, a display section for displaying the outside image captured by the image capturing section, an ocular optical system disposed in front of an eye, having a transparent plate-formed member facing the eye, and a control section that moves the image displayed by the display section from a strabismic line-of-sight direction to the normal line-of-sight direction. The apparatus leads light from the display section to the eye by the ocular optical system to provide the virtual outside image displayed by the display section, and makes light from the outside pass through the plate-formed member to lead the light to the eye to provide a real image of the outside.

This application is based on Japanese Patent Application No. 2004-090475 filed on Mar. 25, 2004, in Japanese Patent Office, the entire content of which is hereby incorporated by reference.

### FIELD OF THE INVENTION

The present invention relates to a virtual image display apparatus for training for correction of strabismus that uses an outside landscape itself.

### BACKGROUND OF THE INVENTION

For training for recovery of visual acuity and visual functions, there is a training system provided with a focal distance adjusting means and depth adjusting means. Such a training system enables training similar to that based on a real image (for example, Patent Document 1). For training for correction of strabismus and recovery of visual acuity, there is also a system that allows a doctor or a patient himself/herself to observe the direction and motion of an eye of the patient in training (for example, Patent Document 2).
[Patent Document 1] TOKKAIHEI No. 7-275286 (pages 1 through 11, FIGS. 1 through 23)
[Patent Document 2] TOKKAIHEI No. 9-38164 (pages 1 through 11, FIGS. 1 through 9)

As described above, there have been offered devices for training for recovery of visual acuity and visual functions. An infant suffering from strabismus (congenital strabismus in most cases), for example, tends to look at an object with its one eye alone. Some infants use the right or left eye, depending on the case, without the working eye being limited to a particular side, while other infants always use the same eye on a fixed side, namely the right or left side.

A problem with the latter case is that restraint is imposed on the eye not used for looking, resulting in that visual acuity does not develop. Further, such an infant may suffer from retarded development of visual function of both eyes, and may become incapable of taking a stereoscopic view. To solve this problem, it is important to remove the restraint imposed on the eye that is not used. Treatment of this problem requires continuous training in daily life. This requires a device that can be taken into a home to be used easily.

In the prior art, an eye patch or the like has been used to cover the working eye and forced an infant to use the eye on which restraint is imposed. This method, however, gives pain to infant patients.

### SUMMARY OF THE INVENTION

To solve this problem, it is an object of the present invention to provide a virtual image display apparatus for training for correction of strabismus that allows easy training for correction of strabismus in daily life, without the need of preparing any particular training content.

In an aspect of the invention, there is provided an apparatus for displaying a virtual image to perform training for correction of strabismus. The apparatus includes an image capturing section for capturing an outside image such that a normal line-of-sight direction is always positioned at a center of the captured image, and a display section for displaying the outside image captured by the image capturing section. The apparatus further includes an ocular optical system disposed in front of an eye, having a transparent plate-formed member facing the eye, and a control section that moves the image displayed by the display section from a strabismic line-of-sight direction to the normal line-of-sight direction. Herein, the apparatus leads light from the display section to the eye by the ocular optical system to provide the eye with the outside image displayed by the display section, the image being a virtual image, and makes light from the outside pass through the plate-formed member to lead the light to the eye to provide the eye with a real image of the outside.

### BRIEF DESCRIPTION OF THE DRAWINGS

FIG. 1 is a structure diagram showing a virtual image display apparatus for training for correction of strabismus;

FIG. 2 is a diagram showing the virtual image display apparatus for training for correction of strabismus fitted to glasses;

FIG. 3 is a cross sectional view including a display section;

FIG. 4 is a plan view of the virtual image display apparatus for training for correction of strabismus; and

FIG. 5 is a flowchart showing steps of training for correction of strabismus by the virtual image display apparatus.

### DETAILED DESCRIPTION OF THE INVENTION

An embodiment of a virtual image display apparatus for training for correction of strabismus in accordance with the present invention will be described below. However, the invention is not limited to it. The present embodiment is the best mode of the invention, and terms related to the invention are not limited to those in the present embodiment.

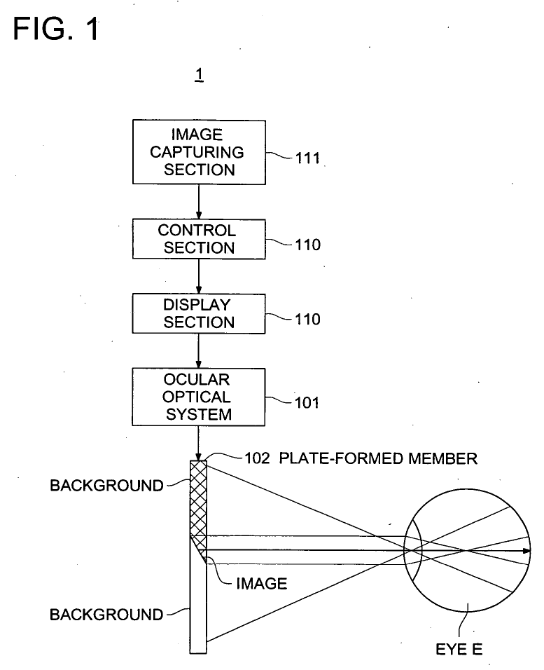

The embodiment of a virtual image display apparatus 1 for training for correction of strabismus is arranged as shown in FIG. 1, which is a structure diagram showing the virtual image display apparatus for training for correction of strabismus.

The virtual image display apparatus 1 for training for correction of strabismus in the present embodiment includes a display section 100 for displaying an image, an ocular optical system 101, and a transparent plate-formed member 102 for holding the ocular optical system 101. The display section 100 is connected with a control section 110, and the control section 110 is connected with an image capturing section 111. The image capturing section 111 includes, for example, a camera using a solid image capturing device such as a CCD camera, and is used to capture an image of a photographic object and to transmit information on the image to the control section 110. The display section 100 is controlled by the control section 110 to display the image. Light from the display section 100 is led to an eye E through the ocular optical system 101 to provide a virtual image displayed by the display section 100. This system also makes light from outside pass through the plate-formed member 102 to lead the light to the eye E so as to provide an image of the outside.

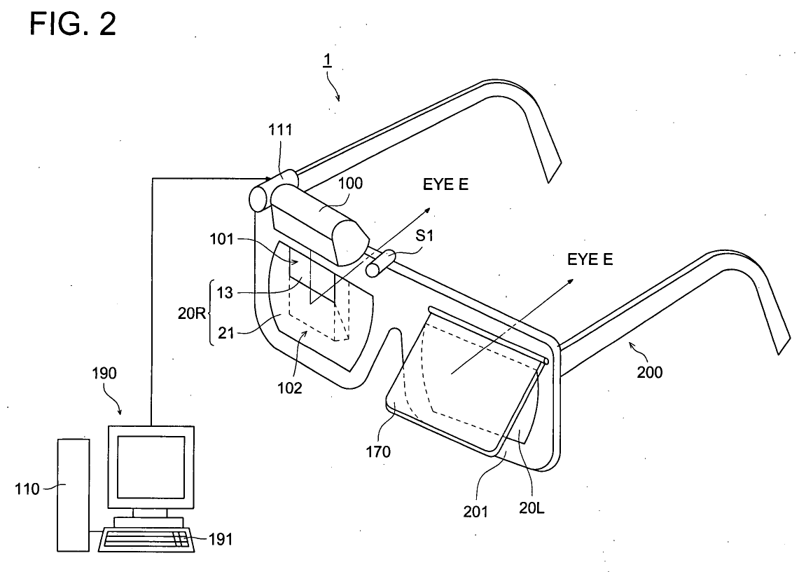

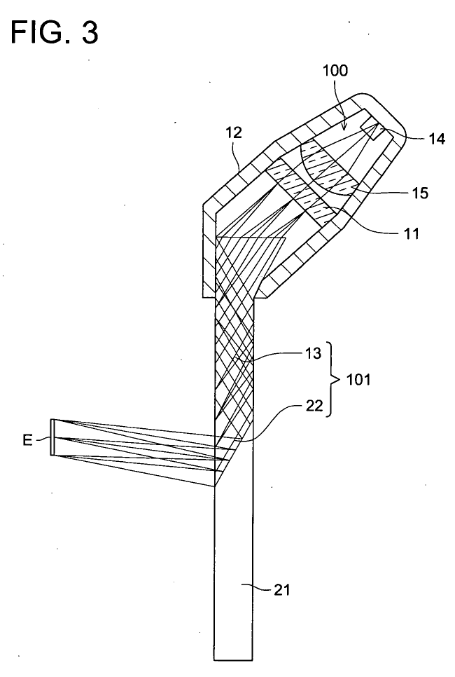

The virtual image display apparatus 1 for training for correction of strabismus in the present embodiment is applicable to glasses, as shown in FIGS. 2 and 3. In the present embodiment, the virtual image display apparatus 1 for training for correction of strabismus has preferably a transmittance rate of 80 percent or higher, and the display section 100 and image capturing section 111 of the virtual image display apparatus 1 are provided on the top of a frame 201 of glasses 200 on the side used for training for correction of strabismus. An open/close cover 170 is provided on the top of the frame 201 on the side not used for training so that the eye E not used for training can be easily covered by the open/close cover 170.

The control section 110 is arranged in a training device 190 for correction of strabismus, which is provided with an operation section 191. Training for correction of strabismus is carried out by operation of the operation section 191.

FIG. 3 is a cross sectional view including the display section. The display section 100 includes a transmission type LCD (liquid crystal display) 11, a casing 12, a prism 13, a LED (Light-Emitting Diode)-14 and a lens 15. The LCD 11 displays an image to be displayed. Illumination light is modulated through the displayed image. The casing 12 accommodates and holds the LCD 11, LED 14 and lens 15. The LED 14 is a light source for illuminating the LCD 11. The lens 15 constructs an illumination optical system that leads light from the LED 14 uniformly over the entire surface of the LCD 11. Without being limited to a transmission type LCD, the display section 100 can also be constructed by a reflection type LCD, for example.

Display of the image by the LCD 11 and emission of the light from the LED 14 are controlled by the control section 110, where the image data captured by the image capturing section 111 is subjected to image processing so that electric power and image signals are supplied.

The prism 13 is a flat plate made of transparent glass or resin. The prism 13 leads light from the LCD 11 to the eye E to display the virtual image displayed on the LCD 11. The top end of the prism 13 is formed in a wedge shape where the edge portion is thicker than the inside portion. The casing 12 is mounted on the prism 13 such that the casing sandwiches this wedge-shaped top end of the prism.

A prism 20L is a flat plate constructed by a single member. A prism 20R is also a flat plate, which is constructed by the prism 13 and a prism 21, and not by a single member. The prism 20L and prism 21 are made of the same material as the prism 13, and these three parts have no difference in refraction index. The prism 13 and prism 21 constructing the prism 20R are formed in a complementary shape without any clearance in-between, and are jointed with each other such that the surfaces are continuous. Except that the prism 13 has a wedge-shaped top end, the prisms 20L and 20R are symmetrical, similarly to commonly used glasses. Thus, a trainee wearing the virtual image display apparatus 1 for training for correction of strabismus on his/her face observes outside through the prisms 20L and 20R.

The bottom end of the prism 13 is formed in a wedge shape in such a manner that the front surface (surface farther from the eye E) becomes closer to the back surface (surface closer to the eye E) as a portion of the prism 13 is closer to the edge. The front surface of this wedge-shaped portion, that is, the joint surface with the prism 21 is a flat plane on which a hologram device 22 is formed. While the trainee is wearing the apparatus, the hologram device 22 is located just in front of the eye E. The prism 13 and hologram device 22 construct the ocular optical system 101.

The prism 13 leads the light from the LCD 11 through the surface of the top end into the inside. The prism 13 leads the light to the hologram device 22 through total reflection of a plurality of times on the front and back surfaces. Diffracting the light having been led, the hologram device 22 forms a luminous flux being almost parallel light beams and leads it into the eye E. Thus, the virtual image displayed on the LCD 11 is displayed to the trainee. The hologram device 22 acts little on the light from outside. The virtual image is observed, overlapping with the central portion of the outside image. The hologram 22 is capable of synthesizing light, and leads light as a mixture of the outside light and the virtual image to the eye E. The transmittance rate of the hologram device 22 is preferably 80 percent or higher. Such a hologram device provides a synthetic image free from a sense of incompatibility to the eye of the trainee. On the other hand, the trainer is allowed to carefully observe the eye of the trainee wearing the virtual image display apparatus 1 for training for correction of strabismus.

The virtual image display apparatus 1 for training for correction of strabismus leads the light from the LCD 11 to the hologram device 22, reflecting the light inside the prism 13. Therefore, the size of the prism 20R can be set large. The bottom end of the prism 13 is formed in a wedge shape. Since it is jointed with the prism 21 made of the same material, there is no refraction of light from outside passing through the bottom end of the prism 13. Thus, the virtual image display apparatus 1 for training for correction of strabismus displays an image in a high quality, without the central portion of the outside image being distorted or discontinuous.

The LCD 11 has a rectangular form with a greater size in the horizontal direction, and can display one to several strings, each string having more than ten characters arranged horizontally. This arrangement allows the trainee to obtain much information at one time from the image. In the present embodiment, an image is displayed to the right eye. Needless to say, it is also possible to display an image to the left eye.

The power of the prism 20R is set according to the visual acuity of the trainee. When the prism 20R is provided with a negative power, myopia is corrected. When the prism 20R is provided with a positive power, hyperopia is corrected. The joint surface of the bottom end of the prism 13 in contact with the prism 21 is formed in a flat shape, and the hologram device 22 can be easily formed. If correction of visual acuity is not required, or if the apparatus is used by a plurality of persons, lens power is not needed. In this case, the prism 21 may be omitted. Incidentally, a virtual image display apparatus 1 for training for correction of strabismus of the right eye is described in the present embodiment, the invention is not limited to this, and can also be applied for a virtual image display apparatus for training for correction of strabismus of the left eye likewise.

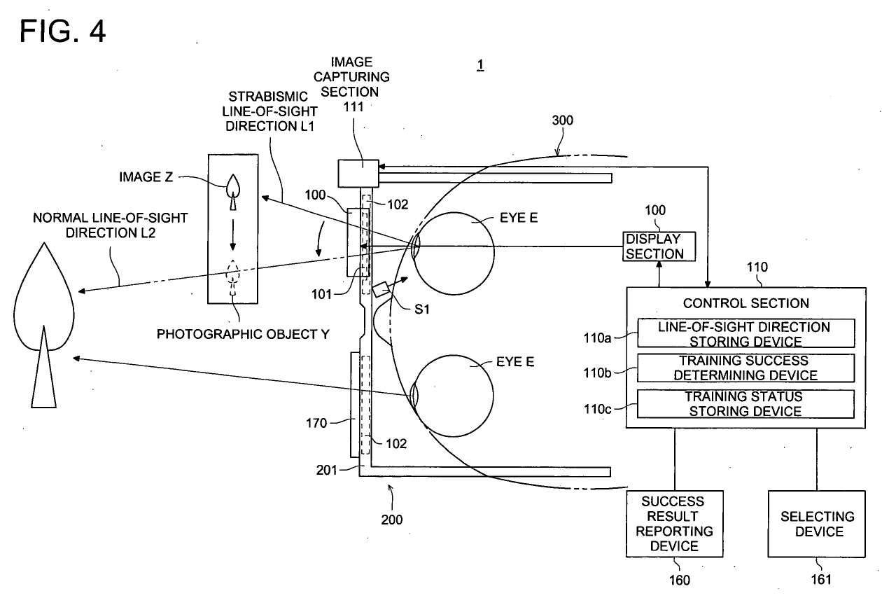

FIG. 4 is a structure diagram of the virtual image display apparatus for training for correction of strabismus. The virtual image display apparatus 1 for training for correction of strabismus in the present embodiment is fitted to the head portion of a trainee 300, who has a normal left eye E and a squint right eye E, as an example. In the virtual image display apparatus 1 for training for correction of strabismus, the image capturing section 111 is capable of capturing an image in such a way that a direction to which an eye is supposed to be normally directed is always positioned at the center of a captured image. Herein, a direction to which an eye is supposed to be normally directed is a normal line-of-sight direction, in other words, a direction to which a normal line-of-sight is supposed to be directed. A direction the image capturing section 111 faces can be fixed in terms of the positional relationship with the frame 201.

The control section 110 controls display in such a way that an image Z displayed on the display section 100 is moved from a strabismic line-of-sight direction L1 to a normal line-of-sight direction L2.

The control section 110 is provided with a line-of-sight direction storing device 110a for storing in advance the normal line-of-sight direction L2. This normal line-of-sight direction L2 is determined by diagnosis in advance and stored, or a normal line-of-sight direction designated by a doctor is stored in advance. Further, the line-of-sight direction storing device 110a also has a function of storing an undesirable line-of-sight direction to which the eye E of the trainee is usually directed.

The control section 110 moves the image Z displayed by the display section 100 from the strabismic line-of-sight direction L1 to the normal line-of-sight direction L2 stored in advance, thereby achieving easy training for correction of the strabismic line-of-sight direction L1 to be the normal line-of-sight direction L2.

The virtual image display apparatus 1 for training for correction of strabismus has a line-of-sight direction detecting device S1 for detecting the strabismic line-of-sight direction L1. This line-of-sight direction detecting section S1 is mounted on the frame 201 of the glasses 200. The line-of-sight direction detecting device S1 detects the strabismic line-of-sight direction, according to the direction of the eye E, and transmits the detected information to the control section 110. The motion of the strabismic eye E can be used to determine success or failure of training for correction of strabismus. A detector for checking the motion of an eyeball by applying infrared rays to the eyeball, for example, can be used as the line-of-sight direction detecting device S1.

In the present embodiment, an outside photographic object is used as a target sign for moving the image Z from a strabismic line-of-sight direction to a normal line-of-sight direction. The control section 110 has a training success determining device 110b. The training success determining device 110b determines success or failure, based on an overlap between a photographic object Y in the normal line-of-sight direction and the photographic object as a target sign of the image Z moving from the strabismic line-of-sight direction to the normal line-of-sight direction.

As described above, an outside photographic object is used as a target sign for moving the image Z from the strabismic line-of-sight direction L1 to the normal line-of-sight direction L2. Success or failure is determined based on the overlap between the photographic object Y in the normal line-of-sight and the photographic object as a target sign moving from the strabismic line-of-sight direction L1 to the normal line-of-sight direction L2. This arrangement allows the landscape of outside itself to be used for training for correction of strabismus, and hence achieves training performable without making the trainee feel boring. Further, success or failure can be determined according to various types of target signs. In determining the success or failure of training, a detection result by the line-of-sight direction detecting device S1 is checked against a record of the normal line-of-sight direction, and the result is regarded as successful when the both directions accord with each other for a preset time period.

The virtual image display apparatus 1 for training for correction of strabismus has a success result reporting device 160 for reporting a result of determination of success. The success result reporting device 160 includes a sound issuing mechanism or an image issuing mechanism. This success result reporting device 160 reports a result of determination of success to the trainee through sound or image, thereby enabling amusing and easy training for correction of strabismus in daily life.

The virtual image display apparatus 1 for training for correction of strabismus has a selecting device 161 for selecting a repeat of training for correction of strabismus or not. The selecting device 161 allows training for correction of strabismus to be performed through easy operation. The selecting device 161 is configured to allow the trainee to select the next operation from, for example, a command menu displayed on a command control panel containing such commands as Start, Next, Repeat and End.

As described above, the image capturing section 111 is capable of capturing an image in such a way that the normal line-of-sight direction is always positioned at the center of the captured image. By moving the image displayed on the display section 100 from a strabismic line-of-sight direction to a normal line-of-sight direction, it is allowed to perform easy training for correction of strabismus in daily life without the necessity of preparing special training contents.

The control section 110 has a training status storing device 110c for storing a status of training for correction of strabismus. The training status storing device 110c stores information on the status of training for correction of strabismus. The information on the status of training for correction of strabismus stored in the control section 110 can be output and fetched into a PC or the like for analysis.

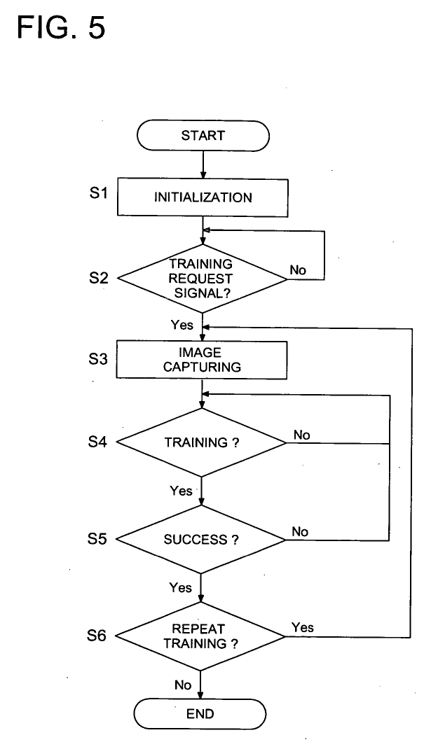

FIG. 5 is a flowchart showing the steps of training for correction of strabismus by the virtual image display apparatus 1. The virtual image display apparatus 1 is fitted to the head portion of the trainee 300 and then power is turned on. Then initialization is performed (Step S1). When a Training button of the operation section is pressed, a training request signal is output (Step S2). Then, an outside photographic object is captured by the image capturing section 111 (Step S3).

The outside image captured by the image capturing section 111 is displayed in the undesirable line-of-sight direction where the eye of the trainee (strabismic) is usually directed. The virtual image display apparatus 1 for training for correction of strabismus is capable of observing outside. Consequently, there is deviation in positional relationship between the landscape of the outside in the correct line-of-sight direction and the center of the image (virtual image) captured by the image capturing section 111, as shown in FIG. 4.

As described above, an image is captured in such a way that the normal line-of-sight direction is always positioned at the center of the captured image. When the image. Z displayed by the display section 100 is moved from the strabismic line-of-sight direction L1 toward the normal line-of-sight direction L2, and the trainee moves the trainee's line-of-sight toward the normal line-of-sight direction, then the motion of the trainee's line-of-sight direction is detected by the line-of-sight direction detecting device S1, and the virtual image displayed on the virtual image display apparatus 1 for training for correction of strabismus follows the motion of the trainee's line-of-sight and moves toward the direction of the trainee's line-of-sight direction. The trainee makes an effort so that the landscape in the normal line-of-sight direction and the landscape at the center of the virtual image overlap with each other (Step S4).

To determine whether the both landscapes overlap with each other or not, the result of detection of the strabismic line-of-sight direction is checked against the record of a normal line-of-sight direction. Then, the success or failure is determined based on the overlap between the photographic object Y in the normal line-of-sight direction and the photographic object as a target sign of the image Z moving from the strabismic line-of-sight direction to the normal line-of-sight direction (Step S5).

When a determination of success is output, an image or voice pleasing an infant may be issued for a predetermined time period (because the trainee is an infant in most cases).

After success or failure has been determined, the trainee can select whether to repeat training or not, and thus can repeat training (Step S6). The position where the line-of-sight of the trainee started moving and the time which was taken to get a successful result are recorded each time training is performed. Further, the motion of the line-of-sight is recorded in a coordinate system. These records can be fetched into a PC for analysis.

The invention includes the following structures.

(1) A virtual image display apparatus for training for correction of strabismus includes an image capturing section for capturing an image of outside, a display section for displaying the image captured by the image capturing section, an ocular optical system, and a transparent plate-formed member for holding the ocular optical system. The virtual image display apparatus is used in such a manner that the ocular optical system is located in front of an eye and the plate-formed member is positioned to face the eye. The virtual image display apparatus leads light from the display section to the eye by the ocular optical system to provide the image displayed by the display section, the image being a virtual image, and makes light from the outside pass through the plate-formed member to lead the light to the eye to provide the image of the outside.

The image capturing section can capture the image such that a normal line-of-sight direction is always positioned at the center of the captured image.

The virtual image display apparatus further includes a control section that moves the image displayed by the display section from a strabismic line-of-sight direction to a normal line-of-sight direction.

Thus, the image capturing section can capture the image such that a normal line-of-sight direction is always positioned at the center of the captured image, and the image displayed by the display section is moved from a strabismic line-of-sight direction to the normal line-of-sight direction. Therefore, it is possible to perform easy training for correction of strabismus in daily life, without the need of preparing any particular training content.

(2) The virtual image display apparatus of above described item (1) for training for correction of strabismus further includes a line-of-sight direction storing device for storing the normal line-of-sight direction in advance, wherein the control section moves the image from the strabismic line-of-sight direction to the normal line-of-sight direction stored in advance.

Thus, it is impossible to perform easy training for correction of a strabismic line-of-sight direction to a normal line-of-sight direction, by moving an image from the strabismic line-of-sight direction to the normal line-of-sight direction stored in advance.

(3) The virtual image display apparatus of item (1) for training for correction of strabismus further includes a line-of-sight direction detecting device for detecting the strabismic line-of-sight direction, and the line-of-sight direction detecting device transmits information on the detected strabismic line-of-sight direction to the control section.

Thus, the motion of a strabismic eye can be used for determination of success of training for correction of strabismus.

(4) With the virtual image display apparatus of item (1) or (2) for training for correction of strabismus uses an outside photographic object as a target sign to move the virtual image from the strabismic line-of-sight direction to the normal line-of-sight direction.

Thus, the landscape of outside itself can be used for training for correction of strabismus, and hence the training can be performed without a trainee getting bored.

(5) The virtual image display apparatus of any one of items (1) to (4) for training for correction of strabismus further includes a training success determining device for determining success of training, based on an overlap between the photographic object in the normal line-of-sight direction and the photographic object as a target sign moving from the strabismic line-of-sight direction to the normal line-of-sight direction.

Thus, success or failure can be determined with various types of target signs, without a trainee getting bored.

(6) In the virtual image display apparatus of item (5) for training for correction of strabismus, the training success determining device checks a detection result by the line-of-sight direction detecting device against a record of the normal line-of-sight direction, and determines that the training is successful when the both directions accord with each other during a preset time period.

Thus, success and failure of training accurate can be determined correctly.

(7) The virtual image display apparatus of any one of items (1) to (6) for training for correction of strabismus further includes a success result reporting device for reporting the result of determination of success.

Thus, the result of determination of success is reported to a trainee, allowing easy training for correction of strabismus in daily life.

(8) The virtual image display apparatus of any one of items (1) to (6) for training for correction strabismus further includes a selecting device for selecting a repeat of the training for correction of strabismus or not.

Thus, training for correction of strabismus can be repeated through easy operation.

(9) The virtual image display apparatus of any one of items (1) to (8) for training for correction of strabismus further includes an open/close cover for covering the eye that is not to be trained for correction of strabismus.

Thus, an eye that is not to be trained for correction of strabismus can be covered easily by an open/close cover.

(10) The virtual image display apparatus of any one of items (1) to (9) for training for correction of strabismus further includes a training status storing device for storing a status of the training for correction of strabismus, wherein information on the status of the training can be output.

Thus, information on the status of training can be output and fetched into a PC or the like for analysis.

The present invention is applicable to virtual image display apparatuses for training for correction of strabismus as follows. A virtual image display apparatus is used in a state where an ocular optical system is located in front of an eye and a plate-formed member faces the eye. The virtual image display apparatus leads light from a display section to the eye through the ocular optical system so as to provide a virtual image displayed by the display section. In addition, the virtual image display apparatus makes light from outside pass through the plate-formed member and leads it to the eye, thereby providing the image of outside.

1. An apparatus for displaying a virtual image to perform training for correction of strabismus, comprising:
an image capturing section for capturing an outside image such that a normal line-of-sight direction is always positioned at a center of the captured image;
a display section for displaying the outside image captured by the image capturing section;
an ocular optical system disposed in front of an eye, having a transparent plate-formed member facing the eye; and
a control section that moves the image displayed by the display section from a strabismic line-of-sight direction to the normal line-of-sight direction, wherein
the apparatus leads light from the display section to the eye by the ocular optical system to provide the eye with the outside image displayed by the display section, the image being a virtual image, and makes light from the outside pass through the plate-formed member to lead the light to the eye to provide the eye with a real image of the outside.

2. The apparatus of claim 1, further comprising a line-of-sight direction storing device for storing the normal line-of-sight direction in advance, wherein the control section moves the virtual image from the strabismic line-of-sight direction to the normal line-of-sight direction stored in advance.

3. The apparatus of claim 1, further comprising a line-of-sight direction detecting device for detecting the strabismic line-of-sight direction, wherein the line-of-sight direction detecting device transmits information on the detected strabismic line-of-sight direction to the control section.

4. The apparatus of claim 1, wherein a photographic object of the outside is employed as a target sign to move the virtual image from the strabismic line-of-sight direction to the normal line-of-sight direction.

5. The apparatus of claim 1, wherein success of training is determined according to an overlap between a real image of a photographic object in the normal line-of-sight direction and a virtual image of the photographic object as a target sign moving from the strabismic line-of-sight direction to the normal line-of-sight direction.

6. The apparatus of claim 5, further comprising a line-of-sight direction detecting device for detecting the strabismic line-of-sight, wherein a direction according to a detection result by the line-of-sight direction detecting device is checked with a record of the normal line-of-sight direction, and when the both directions accord with each other for a preset time period, the training is determined to be successful.

7. The apparatus of claim 5, further comprising a success result reporting device for reporting a result of determination of success.

8. The apparatus of claim 1, further comprising a selecting device for selecting a repeat of training for correction of strabismus or not.

9. The apparatus of claim 1, further comprising an opening and closing cover for covering an eye not to be trained to correct strabismus.

10. The apparatus of claim 1, further comprising a training status storing device for storing a status of training for correction of strabismus, where the information on the status of training for correction of strabismus can be output.
